(19) United States
(12) Patent Application Publication
(10) Pub. No.: US 2022/0347054 A1
(43) Pub. Date: Nov. 3, 2022

(54) DEVICE FOR DELIVERING A FLOWABLE TISSUE DRESSING MATERIAL AND METHODS OF USE

(71) Applicant: KCI Licensing, Inc., San Antonio, TX (US)

(72) Inventors: Timothy Mark ROBINSON, Shillingstone (GB); Christopher Brian LOCKE, San Antonio, TX (US)

(21) Appl. No.: 17/621,142

(22) PCT Filed: Jun. 24, 2020
(86) PCT No.: PCT/US2020/039257

§ 371 Date: Dec. 20, 2021

Related U.S. Application Data
(60) Provisional application No. 62/866,247, filed on Jun. 25, 2019.

(51) Int. Cl. A61J 1/20; A61L 15/14; A61L 15/42; A61L 15/44; A61L 15/22; A61L 15/12

(52) U.S. Cl. CPC A61J 1/20; A61L 15/14; A61L 15/425; A61L 15/44; A61L 15/225; A61L 15/125; A61L 2400/04

(57) ABSTRACT

Devices for delivering a flowable tissue dressing material for treating a tissue site are described. The devices include a first zone including a first reactant, such as a polyol, and a second zone including a second reactant, such as a multi-isocyanate, which can be mixed together to form a flowable tissue dressing material. The device can also include a flowable tissue dressing material including a reacted polymer present in a carrier. Kits and methods including and/or using the devices are also described.

### RELATED APPLICATION

The present application claims priority to U.S. Provisional Patent Application No. 62/866,247, entitled “DEVICE FOR DELIVERING A FLOWABLE TISSUE DRESSING MATERIAL AND METHODS OF USE,” filed Jun. 25, 2019, which is incorporated herein by reference for all purposes.

### TECHNICAL FIELD

The invention set forth in the appended claims relates generally to treatment of tissue, including without limitation to devices for delivering a flowable tissue dressing material to a tissue site, such as a wound, kits, and methods for treating a tissue site.

### BACKGROUND

A wide variety of materials and devices, generally characterized as “dressings,” are generally known in the art for use in treating an injury, defect, or other disruption of tissue. Such disruptions of tissue may be the result of trauma, surgery, or disease, and may affect skin or other tissues. In general, dressings may control bleeding, absorb exudate, ease pain, assist in debriding tissue, protect tissue from infection, or otherwise promote healing and protect tissue from further damage.

Some dressings may protect tissue from, or even assist in the treatment of, infections associated with wounds. Infections can retard wound healing and, if untreated, can result in tissue loss, systemic infections, septic shock and death. While the benefits of dressings are widely accepted, improvements to dressings may benefit healthcare providers and patients.

### SUMMARY

New and useful devices, methods, and kits for treating a tissue site are set forth in the appended claims. Illustrative embodiments are also provided to enable a person skilled in the art to make and use the claimed subject matter.

For example, in some embodiments, a device for delivering a flowable tissue dressing material to a tissue site is described. More generally, a device including a first zone and a second zone is provided. The first zone may include a first reactant, for example, a polyol, a polyaldehyde, or a polyamine. The second zone may include a second reactant, for example, a multi-isocyanate, a multi-isocyanate prepolymer, a polycarbamate, a polycarboxylic acid, or an anhydride. The first zone can be physically separate from the second zone.

In some embodiments, the first zone may be a first container and the second zone may be a second container. In other embodiments, the first zone and the second zone can be present in a single container having a wall defined therein, which separates the first zone and the second zone. The wall can be at least partially removable to allow for mixing between the first reactant and the second reactant to form the flowable tissue dressing material. The flowable tissue dressing material can be capable of solidifying to form an open cell foam when applied to a tissue site.

In some embodiments, the device can further include a third zone for mixing the first reactant with the second reactant to form the flowable tissue dressing material and/or for delivering the flowable tissue dressing material. The third zone is physically separate from the first zone and the second zone.

Alternatively, other example embodiments may include another device for delivering a flowable tissue dressing material to a tissue site. The device includes the flowable tissue dressing material, which includes a reacted polymer present in a carrier. The reacted polymer may be, for example, a polyurethane, a polyester, a polyamide, an acrylic polymer, an acrylate polymer, a polyvinyl acetate, a polysiloxane, or a combination thereof. The carrier may be, for example, a low boiling point liquid, water, a compressed gas, or a combination thereof. The flowable tissue dressing material can be capable of solidifying to form an open cell foam when applied to a tissue site.

Alternatively, other example embodiments may include a kit including a device for delivering a flowable tissue dressing material and a cover. The device may include a first zone and a second zone. The first zone may include a first reactant, for example, a polyol, a polyaldehyde, and a polyamine. The second zone may include a second reactant, for example, a multi-isocyanate, a multi-isocyanate prepolymer, a polycarbamate, a polycarboxylic acid, or an anhydride. The first zone is physically separate from the second zone. Alternatively, the device may include the flowable tissue dressing material, which includes a reacted polymer present in a carrier. The reacted polymer may be, for example, a polyurethane, a polyester, a polyamide, an acrylic polymer, an acrylate polymer, a polyvinyl acetate, a polysiloxane, and a combination thereof. The carrier may be, for example, a low boiling point liquid, water, a compressed gas, or a combination thereof.

Alternatively, other example embodiments may include a method for treating a tissue site. The method can include applying a flowable tissue dressing material from a device to a tissue site, and solidifying the flowable tissue dressing material to form an open cell foam adjacent to the tissue site. The flowable tissue dressing material may be formed in the device by mixing a first reactant with a second reactant to form the flowable tissue dressing material. The first reactant may be, for example, a polyol, a polyaldehyde, or a polyamine. The second reactant may be, for example, a multi-isocyanate, a multi-isocyanate prepolymer, a polycarbamate, a polycarboxylic acid, or an anhydride. Prior to mixing, the first reactant may be present in a first zone and the second reactant may be present in a second zone in the device. The first zone can be physically separate from the second zone. In some embodiments, the first zone may be a first container and the second zone may be a second container. In other embodiments, the first zone and the second zone can be present in a single container having a wall defined therein, which separates the first zone and the second zone. The wall can be at least partially removable to allow for mixing between the first reactant and the second reactant to form the flowable tissue dressing material. In some embodiments, the device can further include a third zone for mixing the first reactant with the second reactant to form the flowable tissue dressing material and/or for delivering the flowable tissue dressing material. The third zone can be physically separate from the first zone and the second zone.

Alternatively, other example embodiments may include another method for treating a tissue site. The method may include applying a flowable tissue dressing material from a device to a tissue site, and solidifying the flowable tissue dressing material to form an open cell foam adjacent to the tissue site. The flowable tissue dressing material includes a reacted polymer present in a carrier. The reacted polymer may be, for example, a polyurethane, a polyester, a polyamide, an acrylic polymer, an acrylate polymer, a polyvinyl acetate, a polysiloxane, or a combination thereof. The carrier may be, for example, a low boiling point liquid, water, a compressed gas, or a combination thereof.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1A is a cross-sectional view, illustrating details that may be associated with some embodiments of a device having a first zone and a second zone.

FIG. 1B is a cross-sectional view, illustrating details that may be associated with some alternative embodiments of a device having a first zone, a second zone, and a third zone.

FIG. 1C is a cross-sectional view, illustrating details that may be associated with some alternative embodiments of a device having a first zone, a second zone, and a third zone.

FIG. 1D is a cross-sectional view, illustrating details that may be associated with some alternative embodiments of a device having a first zone, a second zone, and a canister.

FIG. 1E is a cross-sectional view, illustrating details that may be associated with some alternative embodiments of a device having a first zone, a second zone, and a ultraviolet light source.

FIG. 2 is a cross-sectional view, illustrating details that may be associated with some alternative embodiments of a device having a first zone, a second zone, and a third zone.

FIG. 3 is a cross-sectional view, illustrating details that may be associated with some alternative embodiments of a device having a single container.

### DETAILED DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

### I. Devices for Delivering a Flowable Tissue Dressing Material

Devices for delivering a flowable tissue dressing material for treating a tissue site, for example, for closing and/or filling an opening on a tissue site, such as a wound, are described herein. As used herein, the term “flowable” refers to an ability of a substance to be transported by gravity or under pressure from a storage vessel to a tissue site. Examples of a “flowable” substance include, but are not limited to a liquid, a gel, a slurry, a suspension, an aerosol, and any combination thereof. As used herein, the term “tissue site” broadly refers to a wound or a defect located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. The devices described herein can deliver a flowable tissue dressing material, which can readily conform to the size and shape of the tissue site. Thus, the devices, methods and kits described herein can form tissue dressings in various configurations.

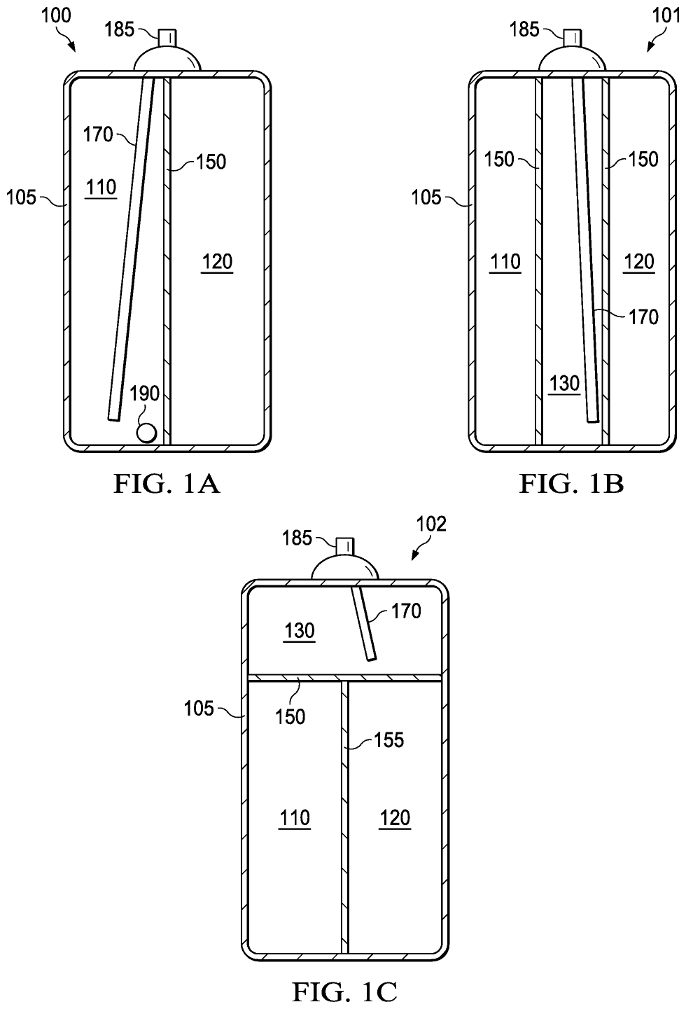

A device for delivering a flowable tissue dressing material may include a first zone comprising a first reactant and a second zone comprising a second reactant. The first zone may be physically separate from the second zone. FIG. 1A illustrates details that may be associated with some embodiments of a device 100. In some embodiments, the device 100 may be a single container 105 including a first zone 110 and a second zone 120 therein. The device 100 can further include a wall 150 defined therein, which separates the first zone 110 and the second zone 120. The wall 150 can be at least partially removable to allow for mixing between the first reactant and the second reactant to form a flowable tissue dressing material upon removal of at least a portion of the wall 150. For example, the wall 150 may be formed of a material, which can be pierced, punctured or removed by a user. Exemplary materials that wall 150 may be formed of include, but are not limited to a metal (e.g., aluminum, steel, and stainless steel), optionally coated with a polymeric coating (e.g., polyurethanes, epoxies, thermosets, such as phenol formaldehyde, urea formaldehyde, melamine formaldehyde, or polyolefins, blends and copolymers thereof), and a polymeric material (e.g., polyamides, acetals, polyesters, and other engineering polymers, such as aramids and aromatic polyesters). The first reactant and the second reactant may be any suitable multipart polymer reaction system, which, when mixed together and/or reacted, form a flowable tissue dressing material, such as a polymer foam, for example, a polyurethane foam. Examples of a suitable first reactant include, but are not limited to a polyol, a polyaldehyde, a polyamine, and combinations thereof. Examples of a suitable second reactant include, but are not limited to a multi-isocyanate (e.g., diisocyanate, triisocyanate), a multi-isocyanate prepolymer, a polycarbamate, a polycarboxylic acid, an anhydride, and combinations thereof. As used herein, the term “multi-isocyanate prepolymer” refers to a multi-isocyanate, such as a diisocyanate, having at least a portion of the active isocyanate groups already reacted leaving fewer isocyanate groups to react with a polyol. For example, the first reactant may be a polyol and the second reactant may be a multi-isocyanate (e.g., diisocyanate). Once the polyol and the multi-isocyanate are mixed together in the presence of water, for example, either present in the device and/or present as ambient water, they can react to form a polyurethane foam, which can be delivered to a tissue site as a flowable tissue dressing material. Similarly, a polyaldehyde (i.e., first reactant) and a polycarbamate (i.e., second reactant) when mixed together can react to form a polyurethane foam.

Optionally, in some embodiments, as illustrated in FIG. 1B, a third zone 130 may be included in a device 101 for mixing the first reactant from the first zone 110 with the second reactant from the second zone 120 to form a flowable tissue dressing material and/or for delivering the flowable tissue dressing material. In the example in FIG. 1B, partially removable walls 150 may be defined therein, which physically separate the first zone 110 and the second zone 120 from the third zone 130. Upon removal of at least a portion of the walls 150, the first reactant and the second reactant may enter the third zone 130 and be admixed to form a flowable tissue dressing material. FIG. 1C illustrates another example configuration of a device 102 having a first zone 110, a second zone 120, a third zone 130 and a wall 150 defined therein, which is at least partially removable. Device 102 further includes an irremovable wall 155 defined therein, which physically separates the first zone 110 and the second zone 120.

Optionally, as illustrated in FIG. 1D, a canister 160 may be in fluid communication with a single container 105 of a device 103. The canister 160 can contain a propellant, as further described below, for further enabling delivery of a flowable tissue dressing material from the device 103. For example, the propellant may expand to force the flowable tissue dressing material out of the device 103, for example, through holes in a spray nozzle as an aerosol. Although not shown, it is contemplated herein that canister 160 can be present in any of the device embodiments described herein. Additionally, the canister 160 may be removable or irremovable. Optionally, as illustrated in FIG. 1E, an ultraviolet (UV) light source 165 may be included with the device 104, for example, for further solidifying a flowable tissue dressing material at a tissue site. While FIG. 1E illustrates a UV light source 165 as integral to the single container 105, it is contemplated herein that the UV light source 165 may be removable from the single container 105 and/or may be separate from the single container 105. Although not shown, it is contemplated herein that UV light source 165 can be present in any of the device embodiments described herein.

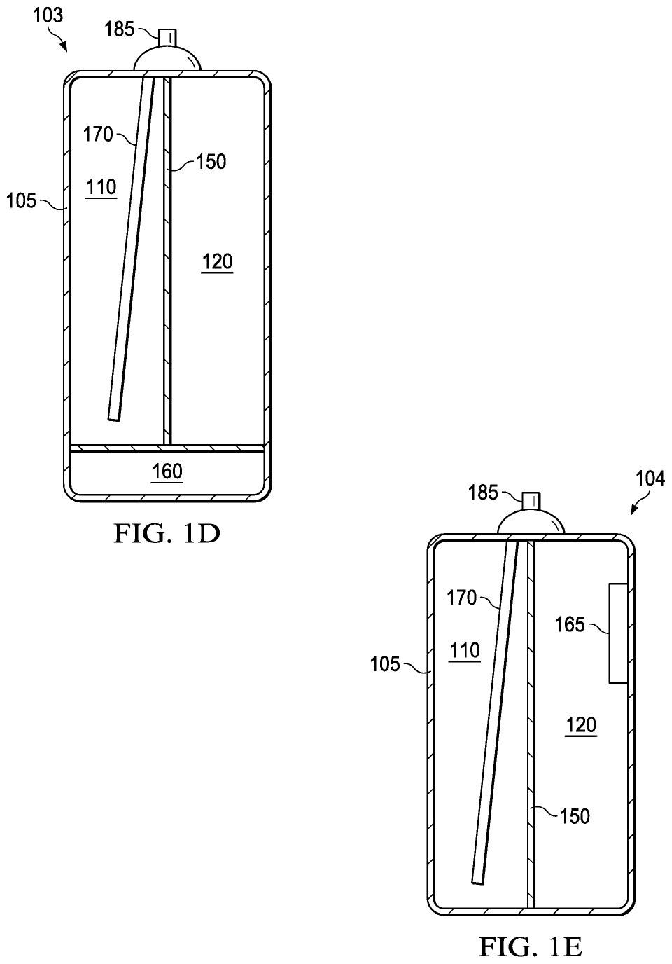

In another example embodiment as illustrated in FIG. 2, a device 200 may include a first zone 110 comprising a first reactant in a first container 210 and a second zone 120 comprising a second reactant in a second container 220. The device 200 may further include a third zone 130 in a third container 230 for combining and/or mixing the first reactant with the second reactant to form a flowable tissue dressing material. For example, the first container 210, the second container 220, and the third container 230 may each include a removable cap 180, so that the first reactant and the second reactant can be removed from the first container 210 and the second container 220 and added to the third container 230. The first reactant and the second reactant may be mixed in any of the first container 210, the second container 220, and the third container 230. It is contemplated herein, if the first reactant and the second reactant are mixed in the first container 210 or the second container 220, the third container 230 may be absent.

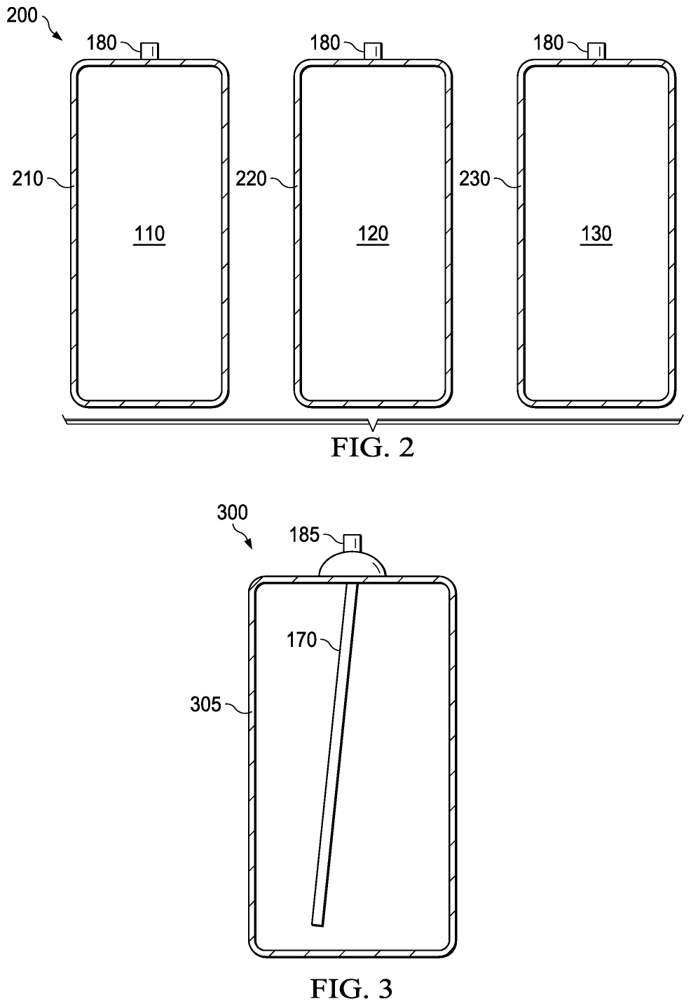

In another example, a device for delivering a flowable dressing material may include the flowable dressing material comprising a reacted polymer present in a carrier. Referring more specifically to FIG. 3, a device 300 contains the flowable dressing material in a single container 305. Examples of a suitable reacted polymer include, but are not limited to a polyurethane, a polyester, a polyamide, an acrylic polymer, an acrylate polymer, a polyvinyl acetate, a polysiloxane, and combinations and copolymers thereof. The reacted polymer may be dissolved or dispersed in a suitable carrier, such as, but not limited to a low boiling point liquid, water, a compressed gas, and combinations thereof. The reacted polymer and carrier may be in the form of a dispersion, solution or emulsion. Examples of a low boiling point liquid include, but are not limited to a fluorocarbon, a chlorofluorocarbon, a hydrofluorocarbon (e.g., tetrafluoropropene, Solkane®) a hydrochlorofluorocarbon, and combinations thereof. Examples of a compressed gas include but are not limited to compressed carbon dioxide, compressed nitrogen, a compressed alkane (e.g., methane, ethane, propane, and the like), and combinations thereof.

As illustrated in FIGS. 1A-1D and 3 a delivery tube 170 optionally may be present for delivering a flowable tissue dressing material from the devices 100, 101, 102, 103, 300. Although not shown in FIG. 2, a delivery tube 170 may be present in one or more of a first container 210, a second container 220, and a third container 230. A delivery means 185 may be in fluid communication with the delivery tube 170 for delivering the flowable dressing material to a tissue site. Examples of suitable delivery means 185 include, but are not limited to a nozzle, such as a spray nozzle, or a manifold delivery tube. In some embodiments, the flowable dressing material may be transferred and/or mixed in a separate vessel (e.g., measuring cup) from which it can be poured onto a tissue site. It is also contemplated herein that a delivery tube 170 may be absent and any of the devices described herein may include a removable cap, so that a flowable tissue dressing material can be poured from the devices onto a tissue site. Optionally, a mixer 190 for mixing the first reactant with the second reactant may be include in the device, for example, as illustrated in FIG. 1A in device 100. Examples of a suitable mixer 190 include but are not limited to a ball (e.g., metal, glass, or plastic ball), a mechanical reciprocating plunger, magnetically coupled impeller or beads, for example, where an external magnetic source rotates the impellor or agitates the beads. Although not shown, it is contemplated herein that the mixer 190 can be present in any of the device embodiments described herein.

The flowable tissue dressing material is capable of solidifying to form a foam when applied to a tissue site. The foam formed may be an open cell foam or a closed cell foam. In any embodiment, the foam may have a higher molecular weight (Mₙ), for example, greater than or equal to about 100,000, greater than or equal to about 500,000 or about 1,000,000; or from about 100,000 to about 1,000,000, about 250,000 to about 1,000,000 or about 500,000 to about 1,000,000. Additionally or alternatively, the foam may have a moisture vapor transmission rate (MVRT) of about 250 g/m²/24 hours to about 1500 g/m²/24 hours, or about 500 g/m²/24 hours to about 1500 g/m²/24 hours, or about 1000 g/m²/24 hours to about 1500 g/m²/24 hours.

In any embodiment, the devices described herein may be made of any suitable material, such as, but not limited to metal, plastic, or a combination thereof. Suitable metals include, but are not limited to aluminum and coated steels. Suitable plastics include, but are not limited to polycarbonates, polyesters, and polyolefins. In any embodiment, an interior of the devices described herein is sterile and the contents of the device may be sterile. Sterilization can be achieved by any known methods in the art, for example, via gamma sterilization or electron beam (e-beam) sterilization. In the case of e-beam sterilization, the devices described herein may include a window, for example, a plastic window, to permit transmission of the e-beam.

The devices described herein can include one or more additional agents for incorporation into a flowable tissue dressing material and/or for use in the formation of a flowable tissue dressing material. Each additional agent may be present in the first zone 110, the second zone 120, the third zone 130, or a combination thereof. In any embodiment, a cell opener can be included in the devices described herein to promote opening or rupturing of cell walls and to enhance an open cell structure as the polymer foam is produced. Examples of a suitable cell opener include, but are not limited to a silicone, a polyether siloxane, a mineral (e.g., clays, silicas, calcium carbonate and the like), and combinations thereof.

Additionally or alternatively, the devices described herein can further include a foaming agent, a propellant, or a combination thereof to assist with foam formation and delivery. As used herein, a foaming agent includes any suitable surfactants and blowing agents as known in the art for producing a flowable tissue dressing material, e.g., a polymer foam. Examples of suitable foaming agents include, but are not limited to a low boiling point liquid, water, a compressed gas, hydrocarbons (e.g. pentane, isopentane, cyclopentane), liquid carbon dioxide, and combinations thereof. Examples of a low boiling point liquid include, but are not limited to a fluorocarbon, a chlorofluorocarbon, a hydrofluorocarbon (e.g., tetrafluoropropene, Solkane®) a hydrochlorofluorocarbon, and combinations thereof. Examples of a compressed gas include but are not limited to compressed carbon dioxide, compressed nitrogen, a compressed alkane (e.g., methane, ethane, propane, and the like), and combinations thereof. Examples of a suitable propellant include, but are not limited to low boiling point liquids as described herein. The propellant may be present within the devices described herein, for example, in the first zone 110, in the second zone 120, in the third zone 130, or a combination thereof, or in the single container 305. Alternatively, with reference to FIG. 1D, the propellant may be present in a separate canister 160 in fluid communication with the devices described herein. In addition to aiding in delivery of a flowable tissue dressing material, the propellant may also aid in mixing of the first reactant with the second reactant or mixing the reacted polymer.

Additionally or alternatively, the devices described herein can further include a catalyst, for example, when the first reactant and the second reactant are present, to assist in formation of a flowable tissue dressing material, e.g., a polymer foam. Any suitable catalysts known in the art for producing polymer foams can be used. For example, suitable gelling catalysts and/or blowing catalysts may be used for forming a polyurethane foam. Examples of catalysts include, but are not limited to, tertiary amine catalysts (e.g., 1,4-diazabicyclo[2.2.2]octane), metal complex catalysts, such as metal carboxylates (e.g., tin carboxylates, bismuth carboxylates, zinc carboxylates, zirconium carboxylates, nickel carboxylates), dibutyltin dilaurate, bismuth octanoate, and platinum catalysts.

Additionally or alternatively, the devices described herein and/or the flowable tissue dressing material can further include a softener, such as water soluble particles, to encourage a certain degree of porosity at the tissue site interface, which upon contact with water present in the wound can soften and/or dissolve to leave pores or fissures in foam. Examples of suitable water soluble particles include, but are not limited to a salt, a water soluble polymer, and combinations thereof. Examples of a salt include, but are not limited to sodium chloride, magnesium chloride, calcium chloride, sodium carbonate, potassium carbonate, and combinations thereof. Examples of water soluble polymers include, but are not limited to polyvinylpyrrolidone (PVP), a polyvinyl alcohol, polyethylene oxide (PEO), carboxy modified polyurethane, hydroxy modified polyurethane, and combinations thereof.

Additionally or alternatively, the devices described herein and/or the flowable tissue dressing material can further include an antimicrobial agent. Examples of suitable antimicrobial agents include, but are not limited to organic acids such as carboxylic acids, silver, gold, zinc, copper, polyhexamethylene biguanide (PHMB), iodine and combinations thereof. Exemplary carboxylic acids include, but are not limited to ascorbic acid (e.g., (R)-3,4-dihydroxy-5-((S)-1,2-dihydroxyethyl)furan-2(5H)-one or Vitamin C), formic acid, gluconic acid, lactic acid, oxalic acid, tartaric acid, peroxy-pyruvic acid, and combinations thereof. Examples of carboxylic acids include, but are not limited to citric acid and acetic acid (i.e., ethanoic acid). The metal (e.g., silver) may be present in metallic form, in ionic form (e.g., a silver salt), or both.

Additionally or alternatively, the devices described herein and/or the flowable tissue dressing material can further include a polysaccharide, such as chitosan and/or an anionic polysaccharide. The anionic polysaccharide may be substantially insoluble in water at pH 7. Additionally or alternatively, the anionic polysaccharide may have a molecular weight greater than about 20,000, more preferably greater than about 50,000. The anionic polysaccharide may be in the form of a film, or fibers having a length greater than 1 mm. Suitable anionic polysaccharides include, but are not limited to, polycarboxylates, alginates, hyaluronates, pectins, carrageenans, xanthan gums, sulfated dextrans, cellulose derivatives, such as carboxymethyl celluloses, and oxidized celluloses. The term “oxidized cellulose” refers to any material produced by the oxidation of cellulose, for example with dinitrogen tetroxide. Such oxidation converts primary alcohol groups on the saccharide residues to carboxylic acid groups, forming uronic acid residues within the cellulose chain. The oxidation generally does not proceed with complete selectivity, and as a result hydroxyl groups on carbons 2 and 3 are occasionally converted to the keto form. These keto units introduce an alkali-labile link, which at pH 7 or higher initiates the decomposition of the polymer via formation of a lactone and sugar ring cleavage. In some embodiments, oxidized cellulose may be oxidized regenerated cellulose (ORC), which may be prepared by oxidation of a regenerated cellulose, such as rayon. It has been known that ORC has haemostatic properties. ORC has been available as a haemostatic fabric called SURGICEL® (Johnson & Johnson Medical, Inc.) since 1950. This product may be produced by the oxidation of a knitted rayon material.

Additionally or alternatively, the devices described herein and/or the flowable tissue dressing material can further include an alcohol, a colorant (e.g., a pigment, a dye), a release agent (e.g., wax, fluorocarbon), and a combination thereof. For example, an alcohol can be included as a further solvent and/or suspending agent along with the reacted polymer. Examples of a suitable alcohol include, but are not limited to ethanol, isopropyl alcohol, and a combination thereof.

Additionally or alternatively, the devices described herein and/or the flowable tissue dressing material can further include a photoinitiator that is capable of undergoing photopolymerization or radiation curing, i.e., producing a free radical when exposed to radiation, e.g., UV light, which can react, for example, with the first reactant and/or the second reactant, to initiate polymer chain growth. Examples of a suitable photoinitiator include, but are not limited to, 2,2-dimethoxy-1,2,-diphenylethan-1-one,1-hydroxy-cyclohexyl-phenyl-ketone (IRGACURE® 184); 1-[4-(2-hydroxyethoxy)-phenyl]-2-hydroxy-2-methyl-1-propane-1-one (IRGACURE® 2959); and 2-benzyl-2-(dimethylamino)-1-[4-(4-morpholinyl) phenyl]-1-butanone (IRGACURE® 369).

### II. Kits for Delivering a Flowable Tissue Dressing Material

Kits including the devices described herein are also provided. The kits may further include a cover. In some embodiments, the cover may provide a bacterial barrier and protection from physical trauma. The cover may also be constructed from a material that can reduce evaporative losses and provide a fluid seal between two components or two environments, such as between a therapeutic environment and a local external environment. The cover may be, for example, an elastomeric film or membrane. The cover may have a high moisture-vapor transmission rate in some applications. For example, the MVTR may be at least 300 g/m² per twenty-four hours in some embodiments. In some example embodiments, the cover may be a polymer drape, such as a polyurethane film, that is permeable to water vapor but impermeable to liquid. Such drapes typically have a thickness in the range of about 25 microns to about 50 microns. For permeable materials, the permeability generally should be low enough that a desired negative pressure may be maintained.

### III. Methods for Treating a Tissue Site

Methods for treating a tissue site with a device as described herein are also provided. The method can include applying a flowable tissue dressing material from a device as described herein to a tissue site and solidifying the flowable tissue dressing material to form a foam as described herein, for example, an open cell foam adjacent to the tissue site. In any embodiment, the flowable tissue dressing material may be poured, injected, or sprayed onto or into a tissue site. In some embodiments, the tissue site is an internal site and the flowable tissue dressing material may be delivered percutaneously.

Solidifying the flowable tissue dressing material can be achieved by any known means in the art, for example, via cooling, reacting, heating, curing, cross-linking, exposure to ultraviolet light, and combinations thereof. In some embodiments, the flowable tissue dressing material is allowed to react completely, e.g., foaming has stopped and the majority of heat is released, before applying the flowable tissue dressing material to a tissue site. In other embodiments, the flowable tissue dressing material is applied to a tissue site while still reacting, e.g., foaming. In such instances, the cooler temperature of the tissue site can slow and/or stop the foaming.

In any embodiment, the flowable tissue dressing material can be formed in a device as described herein by mixing a first reactant as described herein with a second reactant as described herein to form the flowable tissue dressing material. Mixing can be achieved by a user, for example, by partially removing a wall as described herein, e.g., wall 150, to allow the first reactant and the second reactant to mix with one another, and or by agitating the device. Additionally or alternatively, a mixer as described herein, e.g., mixer 190, can be present in the device to aid in the mixing. Prior to mixing, the first reactant can be present in a first zone as described herein, e.g., first zone 110, and the second reactant can be present in a second zone as described herein, e.g., second zone 120, wherein the first zone is physically separate from the second zone. In some embodiments, the devices described herein can further include a third zone as described herein, e.g. third zone 130, for mixing the first reactant with the second reactant to form the flowable tissue dressing material and/or for delivering the flowable tissue dressing material. In other embodiments, a device contains the flowable tissue dressing material, which includes the reacted polymer as described herein present in a carrier as described herein.

The devices, kits, and methods described herein may provide significant advantages. For example, the devices described herein can provide a tissue dressing material that can be readily applied to wounds of varying sizes without needing timely customization. The nature of the flowable tissue dressing material can also allow for better adhesion between the tissue dressing material and skin of a tissue site. Additionally, the devices for delivery of a flowable tissue dressing material can eliminate the need for additional traditional dressing material components, such as support and release layers. Furthermore, the devices are portable and can be used in many environments and settings to produce tissue dressings in various configurations.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the delivery tube 170, the delivery means 185, or both may be eliminated or separated from other components for manufacture or sale.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A device for delivering a flowable tissue dressing material, wherein the device comprises:
a first zone comprising a first reactant selected from the group consisting of a polyol, a polyaldehyde, and a polyamine; and
a second zone comprising a second reactant selected from the group consisting of a multi-isocyanate, a multi-isocyanate prepolymer, a polycarbamate, a polycarboxylic acid, and an anhydride; wherein the first zone is physically separate from the second zone.

2. The device of claim 1, wherein the first zone is a first container and the second zone is a second container.

3. The device of claim 1 or claim 2, wherein the first zone and the second zone are present in a single container having a wall defined therein, which separates the first zone and the second zone, wherein the wall is at least partially removable to allow for mixing between the first reactant and the second reactant to form the flowable tissue dressing material.

4. The device of any one of the previous claims, further comprising a third zone for mixing the first reactant with the second reactant to form the flowable tissue dressing material and/or for delivering the flowable tissue dressing material, wherein the third zone is physically separate from the first zone and the second zone.

5. The device of any one of the previous claim, further comprising one or more of:
(i) a cell opener;
(ii) a foaming agent;
(iii) a catalyst;
(iv) water soluble particles;
(v) a propellant;
(vi) an antimicrobial agent;
(vii) collagen;
(viii) oxidized regenerated cellulose (ORC); and
(ix) chitosan.

6. The device of claim 5, wherein the cell opener is selected from the group consisting of a silicone, a polyether siloxane, a mineral, and a combination thereof.

7. The device of claim 5 or claim 6, wherein the foaming agent is selected from the group consisting of a low boiling point liquid, water, a compressed gas, and a combination thereof.

8. The device of claim 7, wherein the low boiling point liquid is a fluorocarbon, a chlorofluorocarbon, or a combination thereof.

9. The device of claim 7 or claim 8, wherein the compressed gas comprises carbon dioxide, nitrogen, an alkane, or a combination thereof.

10. The device of any one of claims 5 to 9, wherein the water soluble particles comprise a salt, a water soluble polymer, or a combination thereof.

11. The device of claim 10, wherein the salt is selected from the group consisting of sodium chloride, magnesium chloride, calcium chloride, sodium carbonate, potassium carbonate, and a combination thereof.

12. The device of claim 10 or claim 11, wherein the water soluble polymer is selected from the group consisting of polyvinylpyrrolidone (PVP), a polyvinyl alcohol, polyethylene oxide (PEO), carboxy modified polyurethane, hydroxy modified polyurethane, and a combination thereof.

13. The device of any one of claims 5 to 12, wherein the antimicrobial agent is selected from the group consisting of a carboxylic acid, silver, gold, zinc, copper, polyhexamethylene biguanide (PHMB), iodine and a combination thereof.

14. A device for delivering a flowable tissue dressing material, wherein the device comprises:
the flowable tissue dressing material comprising a reacted polymer present in a carrier,
wherein the reacted polymer is selected from the group consisting of a polyurethane, a polyester, a polyamide, an acrylic polymer, an acrylate polymer, a polyvinyl acetate, a polysiloxane, and a combination thereof,
wherein the carrier is a low boiling point liquid, water, a compressed gas, or a combination thereof.

15. The device of claim 14, wherein the low boiling point liquid is a fluorocarbon, a chlorofluorocarbon, or a combination thereof.

16. The device of claim 14 or claim 15, wherein the compressed gas comprises carbon dioxide, nitrogen, an alkane, or a combination thereof.

17. The device of any one of claims 14 to 16, further comprising one or more of:
(i) a cell opener;
(ii) water soluble particles;
(iii) an alcohol;
(iv) a propellant;
(v) an antimicrobial agent;
(vi) collagen;
(vii) oxidized regenerated cellulose (ORC); and
(viii) chitosan.

18. The device of claim 17 wherein the cell opener is selected from the group consisting of a silicone, a polyether siloxane, a mineral, and a combination thereof.

19. The device of claim 17 or 18, wherein the water soluble particles comprises a salt, a water soluble polymer, or a combination thereof.

20. The device of claim 19, wherein the salt is selected from the group consisting of sodium chloride, magnesium chloride, calcium chloride, sodium carbonate, potassium carbonate, and a combination thereof.

21. The device of claim 19 or claim 20, wherein the water soluble polymer is selected from the group consisting of polyvinylpyrrolidone (PVP), a polyvinyl alcohol, polyethylene oxide (PEO), carboxy modified polyurethane, hydroxy modified polyurethane, and a combination thereof.

22. The device of any one of claims 17 to 21, wherein the antimicrobial agent is selected from the group consisting of a carboxylic acid, silver, gold, zinc, copper, polyhexamethylene biguanide (PHMB), iodine and a combination thereof.

23. The device of any one of the previous claims, further comprising one or more of:
(i) a photoinitiator;
(ii) an ultraviolet light source for solidifying the flowable tissue dressing material;
(iii) a mixer for mixing the flowable tissue dressing material; and
(iv) a delivery tube for delivering the flowable tissue dressing material.

24. The device of any one of the previous claims, wherein the flowable tissue dressing material is capable of solidifying to form an open cell foam when applied to a tissue site.

25. The device of claim 24, wherein the open cell foam has a moisture vapor transmission rate of about 250 g/m2/24 hours to about 1500 g/m2/24 hours.

26. The device of claim 24 of claim 25, wherein the open cell foam has a molecular weight of greater than or equal to about 100,000.

27. The device of any one of the previous claims, wherein an interior of the device is sterile and/or the device is formed from metal, plastic, or a combination thereof.

28. A kit comprising the device of any one of the previous claims and a cover.

29. A method for treating a tissue site, the method comprising:
applying a flowable tissue dressing material from a device to a tissue site, wherein the flowable tissue dressing material is formed in the device by mixing a first reactant with a second reactant to form the flowable tissue dressing material, wherein the first reactant is selected from the group consisting of a polyol, polyaldehyde, and a polyamine; and the second reactant is selected from the group consisting of a multi-isocyanate, a multi-isocyanate prepolymer, a polycarbamate, a polycarboxylic acid, and an anhydride; and wherein, prior to mixing, the first reactant is present in a first zone and the second reactant is present in a second zone in the device; wherein the first zone is physically separate from the second zone; and
solidifying the flowable tissue dressing material to form an open cell foam adjacent to the tissue site.

30. The method of claim 29, wherein the first zone is a first container and the second zone is a second container.

31. The method of claim 29 or claim 30, wherein the first zone and the second zone are present in a single container having a wall defined therein, which separates the first zone and the second zone, wherein the wall is at least partially removable to allow for mixing between the first reactant and the second reactant to form the flowable tissue dressing material.

32. The method of any one of claims 29 to 31, wherein the device further comprises a third zone for mixing the first reactant with the second reactant to form the flowable tissue dressing material and/or for delivering the flowable tissue dressing material, wherein the third zone is physically separate from the first zone and the second zone.

33. The method of any one of claims 29 to 32, wherein the device further comprises one or more of:
(i) a cell opener;
(ii) a foaming agent;
(iii) a catalyst;
(iv) water soluble particles;
(v) a propellant
(vi) an antimicrobial agent;
(vii) collagen;
(viii) oxidized regenerated cellulose (ORC); and
(ix) chitosan.

34. The method of claim 33, wherein the cell opener is selected from the group consisting of a silicone, a polyether siloxane, a mineral, and a combination thereof.

35. The method of claim 33 or claim 34, wherein the foaming agent is selected from the group consisting of a low boiling point liquid, water, a compressed gas, and a combination thereof.

36. The method of claim 35, wherein the low boiling point liquid is a fluorocarbon, a chlorofluorocarbon, or a combination thereof.

37. The method of claim 35 or claim 36, wherein the compressed gas comprises carbon dioxide, nitrogen, an alkane, or a combination thereof.

38. The method of any one of claims 33 to 37, wherein the water soluble particles comprises a salt, a water soluble polymer, or a combination thereof.

39. The method of claim 38, wherein the salt is selected from the group consisting of sodium chloride, magnesium chloride, calcium chloride, sodium carbonate, potassium carbonate, and a combination thereof.

40. The method of claim 38 or claim 39, wherein the water soluble polymer is selected from the group consisting of polyvinylpyrrolidone (PVP), a polyvinyl alcohol, polyethylene oxide (PEO), carboxy modified polyurethane, hydroxy modified polyurethane, and a combination thereof.

41. The method of any one of claims 33 to 40, wherein the antimicrobial agent is selected from the group consisting of a carboxylic acid, silver, gold, zinc, copper, polyhexamethylene biguanide (PHMB), iodine and a combination thereof.

42. A method for treating a tissue site, the method comprising:
applying a flowable tissue dressing material from a device to a tissue site, wherein the flowable tissue dressing material comprises a reacted polymer present in a carrier, wherein the reacted polymer is selected from the group consisting of a polyurethane, a polyester, a polyamide, an acrylic polymer, an acrylate polymer, a polyvinyl acetate, a polysiloxane, and a combination thereof, and wherein the carrier is a low boiling point liquid, water, a compressed gas, or a combination thereof; and
solidifying the flowable tissue dressing material to form an open cell foam adjacent to the tissue site.

43. The method of claim 42, wherein the low boiling point liquid is a fluorocarbon, a chlorofluorocarbon, or a combination thereof.

44. The method of claim 42 or claim 43, wherein the compressed gas comprises carbon dioxide, nitrogen, an alkane, or a combination thereof.

45. The method of any one of claims 42 to 44, wherein the device further comprising one or more of:
(i) a cell opener;
(ii) water soluble particles;
(iii) an alcohol;
(iv) a propellant;
(v) an antimicrobial agent;
(vi) collagen;
(vii) oxidized regenerated cellulose (ORC); and
(viii) chitosan.

46. The method of claim 45, wherein the cell opener is selected from the group consisting of a silicone, a polyether siloxane, a mineral, and a combination thereof.

47. The method of claim 45 or claim 46, wherein the water soluble particles comprises a salt, a water soluble polymer, or a combination thereof.

48. The method of claim 47, wherein the salt is selected from the group consisting of sodium chloride, magnesium chloride, calcium chloride, sodium carbonate, potassium carbonate, and a combination thereof.

49. The method of claim 47 or claim 48, wherein the water soluble polymer is selected from the group consisting of polyvinylpyrrolidone (PVP), a polyvinyl alcohol, polyethylene oxide (PEO), carboxy modified polyurethane, hydroxy modified polyurethane, and a combination thereof.

50. The method of any one of claims 45 to 49, wherein the antimicrobial agent is selected from the group consisting of a carboxylic acid, silver, gold, zinc, copper, polyhexamethylene biguanide (PHMB), iodine and a combination thereof.

51. The method of any one of claims 29 to 50, wherein the device further comprises one or more of:
(i) a photoinitiator;
(ii) an ultraviolet light source for solidifying the flowable tissue dressing material;
(iii) a mixer for mixing the flowable tissue dressing material; and
(iv) a delivery tube for delivering the flowable tissue dressing material.

52. The method of any one of claims 29 to 51, wherein the open cell foam has a moisture vapor transmission rate of about 250 g/m2/24 hours to about 1500 g/m2/24 hours.

53. The method of any one of claims 29 to 52, wherein the open cell foam has a molecular weight of greater than or equal to about 100,000.

54. The method of any one of claims 29 to 53, wherein an interior of the device is sterile and/or the device is formed from metal, plastic, or a combination thereof.
